(19) United States
(12) Patent Application Publication
(10) Pub. No.: US 2021/0101028 A1
(43) Pub. Date: Apr. 8, 2021

(54) Endure, Anti-radiation supplement

(71) Applicant: Tim Chrisman, Falls Church, VA (US)

(72) Inventor: Tim Chrisman, Falls Church, VA (US)

(73) Assignee: Tim Chrisman, Falls Church, VA (US)

(21) Appl. No.: 17/122,328

(22) Filed: Dec. 15, 2020

(51) Int. Cl. A61P 17/16

(52) U.S. Cl. CPC A61P 17/16; A23V 2002/00

(57) ABSTRACT

Endure is a nutritional supplement designed to slow the negative effects of radiation on the brain, eyes and intestines while an individual is traveling in the upper atmosphere or outer space. It combines 6 natural compounds shown in clinical trials to reduce radiation-induced inflammation, into a single supplement.

### ENDURE COMPOSITION AND USE

### FORMULATION

Ingredients are combined into two tablets
500 mg Vitamin C
500 mg Zingerone
300 IU Vitamin E
100 μg Selenium
5 mg Lutein
1 mg Zeaxanthin

Supplement is to be take two times daily for two weeks prior to spaceflight, and three times daily during any extended flights off planet to help relieve the negative effects of radiation on the brain, eyes and intestines.

1. A tablet or capsule consisting essentially of not less than 225 mg and not more than 275 mg Vitamin C extract (as ascorbic acid), not less than 225 mg and not more than 275 mg Zingerone (also known as vanillylacetone), not less than 125 International Units (IU) and not more than 175 IU of Vitamin E, not less than 2 mg and not more than 3 mg Lutein, not less than 450 μg and not more than 550 μg Zeaxanthin and not less than 40 μg and not more than 60 μg Selenium.
